(19) United States
(12) Patent Application Publication
(10) Pub. No.: US 2009/0022726 A1
(43) Pub. Date: Jan. 22, 2009

(54) METHOD OF USING IL6 ANTAGONISTS WITH PROTEASOME INHIBITORS

(76) Inventors: Mohamed Zaki, Audabon, PA (US); Jeffrey Nemeth, Drexel Hill, PA (US); Robert Z. Orlowski, Chapel Hill, NC (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/608,342

(22) Filed: Dec. 8, 2006

Related U.S. Application Data
(60) Provisional application No. 60/749,152, filed on Dec. 9, 2005.

(51) Int. Cl. A61K 39/395; A61K 31/69; A61K 31/407; A61K 31/4025; A61K 31/336

(52) U.S. Cl. 424/141.1; 424/130.1; 514/64; 514/413; 514/422; 514/475

(57) ABSTRACT

The invention is directed to a method of treating a cancerous disorder or condition, or an IL-6related disorder or condition, in a mammal in need of such treatment, which comprises co-administering a proteasome inhibitor in combination with an IL-6antagonist

### CROSS REFERENCE TO RELATED APPLICATION

This application claims priority to provisional application Ser. No. 60/749,152 filed Dec. 9, 2005.

### BACKGROUND OF THE INVENTION

1. Field of the Invention

The present invention relates to the use of a proteasome inhibitor in combination with an interleukin-6 antagonist to enhance the response of treatment of a subject being treated for diseases, such as cancer. The present invention also relates to methods for treating cancer in a subject by administering to a subject an effective amount of a proteasome inhibitor and an effective amount of an interleukin-6 antagonist. The present invention particularly relates to antibodies, including specified portions or variants, specific for Interleukin-6 (IL-6 also known as Interferon β2)) protein.

2. Background

### Cytokine IL-6

IL-6 (interleukin 6) is a 22-27 kDa secreted glycoprotein formerly known as monocyte-derived human B-cell growth factor, B-cell stimulatory factor 2, BSF-2, interferon beta-2, and hybridoma growth factor, which has growth stimulatory and proinflammatory activities (Hirano et al. Nature 324: 73-76, 1986).

IL-6 belongs to the granulocyte colony-stimulating factor (G-CSF) and myelomonocytic growth factor (MGF) family which includes leukemia inhibitory factor (LIF), oncostatin M (OSM), ciliary neurotropic factor (CNTF), cardiotropin-1 (CT-1), IL-1, and IL-11. IL-6 is produced by an array of cell types, most notably antigen presenting cells, T cells and B cells. IL-6-type cytokines all act via receptor complexes containing a common signal transducing protein, gp130 (formerly IL-6Rbeta). However, whereas IL-6, IL-11, CT-1, and CNTF bind first to specific receptor proteins which subsequently associate with pg130, LIF and OSM bind directly to a complex of LIF-R and gp130. The specific IL-6 receptor (IL-6R or IL-6alpha, gp80, or CD126) exists in either membrane bound or soluble forms (sIL-6R, a 55 kD form), which are both capable of activating gp130.

Several agents are known to induce the expression of IL-6 such as IL-1, IL-2, TNFa, IL-4, IFNa, oncostatin and LPS. IL-6 is involved in diverse activities such as B and T cell activation, hematopoiesis, osteoclast activity, keratinocyte growth, acute phase protein synthesis, neuronal growth and hepatocyte activation (Hirano et al. Int. Rev. Immunol; 16(3-4):249-84, 1998).

Although IL-6 is involved in many pathways, IL-6 knockout mice have a normal phenotype, they are viable and fertile, and show slightly decreased number of T cells and decreased acute phase protein response to tissue injury (Kopf M et al. Nature: 368:339-42, 1994). In contrast, transgenic mice that over-express cerebral IL-6 develop neurologic disease such as neurodegeneration, astrocytosis, cerebral angiogenesis, and these mice do not develop a blood brain barrier (Campbell et al. PNAS 90: 10061-10065, 1993).

### The Role of IL-6 in Cancer

IL-6 is implicated in the pathophysiology of several malignant diseases by a variety of mechanisms. IL-6 is hypothesized to be a causative factor in cancer-related morbidity such as asthenia/cachexia and bone resorption. Tumor-induced cachexia (Cahlin et al. (2000) Cancer Res; 60(19):5488-9), bone resorption and associated hypercalcemia were found to be diminished in IL-6 knockout mice (Sandhu et al. 1999). Cancer-associated depression, and cerebral edema secondary to brain tumors have also been associated with high levels of IL-6 (Musselman et al. Am J Psychiatry.; 158(8):1252-7, 2001).

Experimental results from a number of in vitro and in vivo models of various human cancers have demonstrated that IL-6 is a therapeutic target for inhibition. IL-6 can induce proliferation, differentiation and survival of tumor cells, promote apoptosis (Jee et al. Oncogene 20: 198-208, 2001), and induce resistance to chemotherapy (Conze et al. Cancer Res 61: 8851-8858, 2001).

Multiple myeloma is malignancy involving plasma cells. IL-6 is known to enhance proliferation, differentiation and survival of malignant plasma cells in multiple myeloma (MM) through an autocrine or a paracrine mechanism that involves the inhibition of apoptosis of the malignant cells. Accordingly, blocking of IL-6 has been postulated to be an effective therapy (Anderson et al. Hematology: 147-165, 2000). Both in vitro experiments (Tassone, P. et al. Int. J. Oncol. 21(4): 867-873, 2002) and clinical trials have been performed (Bataille et al. (1995) Blood; 86(2):685-91 and Van Zaanen, et al. (1996) J Clin Invest 98: 1441-1448) and the results indicate that IL6 blockade has demonstrable effect on cancer cell growth.

### The Proteasome Pathway as Therapeutic Target

Recent experimental evidence strongly suggests that proteasome inhibitors may indeed be beneficial in certain pathologies, such as in cancer, asthma, brain infarct, and autoimmune encephalomyelitis. In malignancies, the drugs may act via inhibition of degradation of different cell cycle inhibitors or via inhibition of the anti-apoptotic transcriptional regulator NF-κB, whereas in neuroprotection they may act via inhibiting activation of NF-κB, which in this case elicits the inflammatory response. In autoimmune diseases, they may act by inhibiting presentation of “self” peptides, but also by interfering with signal transduction along cellular immune cascades.

The boronic acid dipeptide proteasome inhibitor PS-341, bortezomib (VELCADE®), is the first approved therapeutic known to act as a potent and specific proteasome inhibitor. Although bortezomib is an important advance in the treatment of myeloma, only 27% of patients with refractory or relapsed disease had partial responses or better in the initial phase II clinical trial that led to its FDA approval (Richardson P G et al, N Engl J Med 2003, 348: 2609-17). Pre-clinical studies have identified important mediators of inducible chemoresistance, including anti-apoptotic pathways which are upregulated upon exposure to proteasome inhibitors, thereby attenuating their anti-tumor efficacy (reviewed in Saleh A et al, Nat Cell Biol 2000, 2: 476-83).

One mechanism of chemoresistance to proteasome inhibitors is the induction of expression of HSP-70, an important inhibitor of apoptosis. Inhibition of the proteasome leads to the accumulation of misfolded proteins and a dramatic up-regulation of members of the heat shock protein family, most notably HSP-70, through activation of the transcription factor, HSF-1.5-8 It was hypothesized that therapeutics that abrogate induction of HSP-70 should be able potentiate the activity of proteasome inhibitors. In other studies, down-regulation of HSP-70 expression through siRNA or anti-sense techniques potentiated the pro-apoptotic activity of proteasome inhibitors in other pre-clinical models of cancer (Robertson J D et al, Biochem J 1999, 344: 477-85; Gabai V L et al, Oncogene 2005, 24: 3328-38).

A second example of inducible chemoresistance is the MKP-1 phosphatase, which is transcriptionally up-regulated by proteasome inhibitors (Orlowski R Z et al, J Biol Chem 2002, 277: 27864-71). MKP-1 phosphatase is a stress response protein which is also anti-apoptotic, acting by inactivation of c-Jun-N-terminal kinase. Down-regulation of MKP-1 has been shown to enhance the anti-tumor efficacy of proteasome inhibitors (Small G W et al, Mol Pharmacol 2004, 66: 1478-90).

IL-6 plays a central role in the pathogenesis of myeloma as demonstrated by its ability to function as a growth and survival factor for myeloma cells in the bone marrow microenvironment, and to activate an anti-apoptotic program that decreases sensitivity to a variety of chemotherapeutics. IL-6 has been shown to up-regulate the expression of HSP-70 in several model systems. Secondly, STAT-1, an important downstream transcription factor activated by IL-6 signaling, interacts with HSF-1 to promote transcription of members of the heat shock response.

Therefore, in the search for more efficacious, less toxic, and more durable clinical responses it would be important to demonstrate combinations of agents valuable in treating certain cancers and related or unrelated muscle wasting, as well as certain inflammatory or autoimmune disorders of neural or nonneural origin. The advantageous effects of combining cytokine of IL-6 inhibitors and proteosome inhibitors has heretofore not been demonstrated.

### SUMMARY OF THE INVENTION

The present invention relates to methods for treating disease in a subject by administering to a subject an effective amount of a proteasome inhibitor and an effective amount of an interleukin-6 antagonist. The method of the invention comprises administration of an anti-IL6 antagonist sequentially, serially, or concurrently with bortezomib or related proteosome inhibitors. In one embodiment, the IL6 antagonist is a high affinity anti-IL6 antibody. In another embodiment, the IL6 antagonist is an anti-IL6R antibody.

A disease amenable to the method of the invention includes cancer, asthma, inflammatory disease and neurological disease. In one embodiment, the disease is a cancerous disorder or condition.

The present invention further provides a method for predicting the utility of a combination of at least IL-6 antagonist and at least one proteosome inhibitor.

### BRIEF DESCRIPTION OF THE DRAWINGS

FIG. 1 is graph showing the effect of increasing concentration of CNT0328 on multiple myeloma cells treated for the indicated times.

FIG. 2 A-D are column graphs representing the relative percent viability of the indicated cells incubated with antibody, F105 and irrelevant control Mab or CNT0328 and the indicated concentration of bortezomib: A) ANBL-6 multiple myeloma cells pre-incubated with antibody and then treated with bortezomib at the indicated concentration, B) KAS-6 multiple myeloma cells pre-incubated with antibody and then treated with bortezomib at the indicated concentration, c) RPMI8226 IL6 independent myeloma cells, and D) ANBL-6 multiple myeloma cells treated concurrently with CNTO 328 and bortezomib.

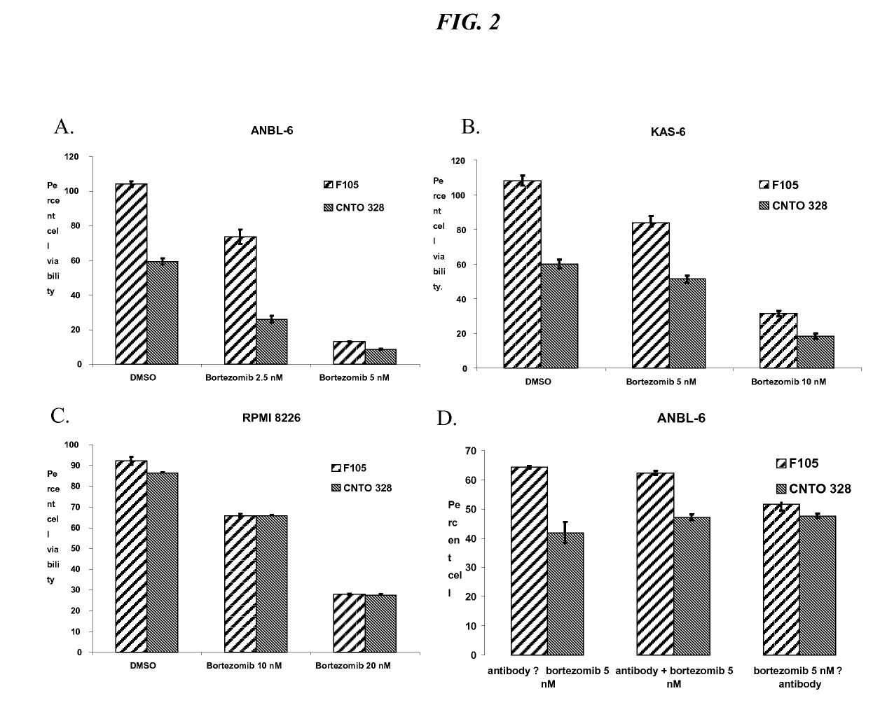

FIG. 3A-B are column graphs representing the relative fold increase in apoptosis measured in the IL6 dependent cell lines ANBL-6 (A) and KAX-6 (B) treated with antibody and bortezomib combinations where F105 is the control Mab.

FIG. 4 is a Western blot of a protein gel of ANBL-6 cells samples after treatment with CNTO328 or control Mab and increasing concentrations of bortezomib probed for HSC-70 and MKP-1.

FIG. 5 is a column graph representing the relative fold increase in apoptosis measured in ANBL-6 cells incubated with the carrier control (DMSO) or two concentrations of bortezomib and increasing concentrations of the heat shock protein attenuator KNK437.

FIG. 6 is a column graph showing the relative fold increase in apoptosis measured in MEF cells which are HSF-deficient (−/−) or normal (+/+) incubated with the carrier control (DMSO) or two concentrations of bortezomib and increasing concentrations of the heat shock protein attenuator KNK437.

### DETAILED DESCRIPTION OF THE INVENTION

### Abbreviations

Ig immunoglobulin, IgG immunoglobulin G, IL interleukin, IL6 interleukin-6, IL-6R interleukin-6 receptor, sIL-6R soluble interleukin-6 receptor, HSF-1 heat shock transcription factor, HSP heat shock protein, MAPK mitogen activated protein kinase, MPK-1 MAPK phosphatase, Mab monoclonal antibody, STAT signal transduction activation

### Definitions

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibody fragments” comprise a portion of a full length antibody, generally the antigen binding or variable domain thereof. Examples of antibody fragments include Fab, Fab′, F(ab′)2, and Fv fragments; diabodies; linear antibodies; single-chain antibody molecules; and multispecific antibodies formed from antibody fragments.

“Chimeric antibodies” are those antibodies that retain distinct domains, usually the variable domain, from one species and the remainder from another species; e.g. mouse-human chimeras.

The term “human antibody”, as used herein, is intended to include antibodies having variable and constant regions derived from or closely matching human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo such as during the recombination of V, D, and J segments of the human heavy chain). Thus as used herein, the term “human antibody” refers to an antibody in which substantially every part of the protein (e.g., CDR, framework, CL, CH domains (e.g., C_{H}1, C_{H}2, C_{H}3), hinge, (V_{L}, V_{H})) is substantially similar to those encoded by human germline antibody genes. Human antibodies have been classified into groupings based on their amino acid sequence similarities, see e.g. http://people.cryst.bbk.ac.uk/˜ubcg07s/. Thus, using a sequence similarity search, an antibody with similar linear sequence can be chosen as a template to select or create human or humanized antibodies.

As used herein, the term “high affinity” for an antibody refers to an antibody having a K_{D} Of 10⁻⁸ M or less, more preferably 10⁻⁹ M or less and even more preferably 10⁻¹⁰ M or less. The term “Kdis” or “K_{D},” or “Kd” as used herein, is intended to refer to the dissociation rate of a particular antibody-antigen interaction. The “K_{D}”, is the ratio of the rate of dissociation (k₂), also called the “off-rate (k_{off})”, to the rate of association rate (k₁) or “on-rate (kₒₙ)”. Thus, K_{D} equals k2/k1 or k_{off}/kₒₙ and is expressed as a molar concentration (M). It follows that the smaller Kd, the stronger the binding. So 10⁻⁶M (or 1 mM) indicates weak binding compared to 10⁻⁹ M (or 1 nM).

As used herein, the “ubiquitin-proteasome system” is a multi-component system that; identifies and degrades unwanted proteins. The system includes the enzymes required for recognizing the unwanted proteins due to their damage, misfolding or short lived cellular nature, which are enzymes related to ubiqutinylation of the unwanted proteins as well as the degradative enzymes which comprise the proteasome structure which is a multisubunit complex found in both the nucleus and cytosol.

As used herein, the term “proteasome inhibitor” is intended to include inhibitors of the peptidases of the proteasome. More specifically, these inhibitors of the peptidases of the proteasome include inhibitors of the chymotrypsin-like and trypsin-like proteases, in addition to thiol and serine proteases.

As used herein, the term “resistant” to a therapeutic agent when referring to a cancer cell means that the cell has achieved resistance to the effects of the agent normally caused by exposure to a environmental level or concentration of that agent with impairs or inhibits proliferation, or is inhibited to a very low degree, as a result of contact with the level of therapeutic agent when compared to a when normal or nonresistant cells are brought in contact with the same level or concentration of the therapeutic agent. The quality of being resistant to a therapeutic agent is a highly variable one, with different cancer cells exhibiting different levels of “resistance” to a given therapeutic agent under different conditions.

The proteasome inhibitor bortezomib represents a significant advance in the treatment of multiple myeloma, but its efficacy is limited by a number of resistance mechanisms. One of the most important is the heat shock protein (HSP) and stress response pathways which, through members such as HSP-70 and mitogen-activated protein kinase (MAPK) phosphatase (MKP)-1, oppose the pro-apoptotic activities of bortezomib. Because interleukin (IL)-6 signaling augments the heat shock response through signal transducer and activator of transcription (STAT)-1 and heat shock transcription factor (HSF)-1, applicants hypothesized that downregulation of IL-6 signaling would attenuate HSP induction by bortezomib, thereby enhancing its anti-myeloma activity.

Treatment of the IL-6-dependent multiple myeloma cell lines KAS-6 and ANBL-6 with the combination of bortezomib and CNTO 328, a chimeric monoclonal IL-6 neutralizing antibody, resulted in greater reduction of cell viability than with either drug alone in a time- and concentration-dependent manner. This was associated with an enhanced induction of apoptosis which, under some conditions, was greater than the sum of the two individual agents alone, suggesting a synergistic interaction. Similar findings were not seen when using isotype control antibodies, and in studies of the IL-6-independent RPMI 8226 myeloma cell line. Increased activity was seen when cells were pre-treated with CNTO 328 followed by bortezomib, or when they were treated with both agents concurrently, compared to treatment with bortezomib followed by CNTO 328. Treatment with CNTO 328 potently inhibited IL-6-mediated downstream signaling pathways, as demonstrated by marked blockade of STAT-3 and p44/42 MAPK phosphorylation. CNTO 328 decreased bortezomib-mediated induction of HSP70 and MKP-1 expression by 45% and 90%, respectively. Notably, CNTO 328 markedly reduced levels of transcriptionally active phospho-STAT-1 and decreased hyperphosphorylation of HSF-1. Other strategies to suppress the heat shock response, including the use of the pharmacologic inhibitor KNK437, also yielded evidence for a synergistic anti-myeloma effect in combination with bortezomib. The synergistic activity of KNK437 and bortezomib was reproduced in normal mouse embryo fibroblasts (MEFs), but blunted in HSF-1 knockout MEFs. Taken together, applicants have demonstrated that inhibition of IL-6 signaling enhances the anti-myeloma activity of bortezomib. They also support the hypothesis that this occurs, at least in part, by attenuating proteasome inhibitor-mediated induction of the heat shock response through down-regulation of transcriptionally active STAT-1 and HSF-1. The teachings of the instant invention provide a rationale for the method of treating a subjects in need thereof with the anti-IL6 antibodies in sequentially, serially, or concurrently with bortezomib or related proteosome inhibitors.

IL6 Antagonists of the Invention

The IL-6 antagonist used in the present invention may be of any origin provided it blocks signal transmission by IL-6, and inhibits the biological activity of IL-6. Examples of IL-6 antagonists include IL-6 antibody, IL-6R antibody, gp130 antibody, IL-6 mutant, IL-6R antisense oligonucleotide, and partial peptides of IL-6 or IL-6R. An example of the IL-6 mutant used in the present invention is disclosed in Brakenhoff, et al., J. Biol. Chem., 269, 86-93, 1994 or Savino, et al., EMBO J., 13, 1357-1367, 1994. The IL-6 mutant polypeptide or fragment thereof does not possess the signal transmission effects of IL-6 but retains the binding activity with IL-6R, and is produced by introducing a mutation in the form of a substitution, deletion or insertion into the amino acid sequence of IL6. While there are no limitations on the animal species used, it is preferable to use an IL6 of human origin. Similarly, any IL-6 partial peptides or IL-6R partial peptides used in the present invention provided they prevent IL6 or IL6R (gp80) or gp130 from affecting signal transduction and thereby prevent IL-6 associated biological activity (U.S. Pat. No. 5,210,075; EP617126 for details regarding IL-6 partial peptides and IL-6R partial peptides). In yet another embodiment, oligonucleotides capable of IL6 or IL6R RNA silencing or antisense mechanisms can be used in the method of the present invention (JP5-300338 for details regarding IL-6R antisense oligonucleotide).

### ANTIBODIES OF THE INVENTION

Antibodies useful in the present invention include isolated chimeric, humanized and/or CDR-grafted, or human antibodies, having at least one antigen binding region which are capable of inhibiting the biological functions of IL6. Examples of antibodies of the invention include IL-6 binding antibody, IL-6R (gp80) binding antibody, gp130-binding antibody. Examples of IL-6R antibodies with suitable antigen binding regions include PM-1 antibody (Hirata, et al., J. Immunol., 143, 2900-2906, 1989), and AUK12-20, AUK64-7 or AUK146-15 antibody (WO92-19759). In another embodiment, the anti-IL6R antibody is the reshaped antibody known as MRA disclosed in U.S. Pat. Nos. 5,888,510 and 6,121,423.

In one embodiment the antigen binding region is derived from the high affinity CLB-8 anti-IL-6 antibody. An exemplary antibody of the invention derived from CLB-6 is CNTO328 as described in applicants co-pending application U.S. Ser. No. 10/280716 the contents of which are incorporated herein by reference. In an alternate embodiment, the antibody is a human antibody which binds IL6 with high affinity such as is described in applicants co-pending U.S. provisional patent application Ser. No. 60/677,319. The antibody of the invention specifically neutralizes human IL-6 with high affinity.

An anti-IL-6 antibody which may be used in the method according to the present invention includes any protein or peptide molecule that comprises at least one complementarity determining region (CDR) of a heavy or light chain or a ligand binding portion thereof, derived from the murine CLB-8 monoclonal antibody, in combination with a heavy chain or light chain constant region, a framework region, or any portion thereof, that can be incorporated into an antibody of the present invention. In one embodiment the invention is directed to an anti-IL-6 chimeric antibody comprising two light chains and two heavy chains, each of the chains comprising at least part of a human constant region and at least part of a variable region (v) derived from the murine c-CLB8 monoclonal antibody having specificity to human IL-6, said antibody binding with high affinity to an inhibiting and/or neutralizing epitope of human IL-6, such as the antibody cCLB-8. The invention also includes fragments or a derivative of such an antibody, such as one or more portions of the antibody chain, such as the heavy chain constant, joining, diversity or variable regions, or the light chain constant, joining or variable regions.

Preferred antibodies of the present invention include those chimeric, humanized and/or CDR grafted, or human antibodies that will competitively inhibit in vivo binding to human IL-6 of anti-IL-6 murine CLB-8, chimeric anti-IL-6 CLB-8, or an antibody having substantially the same binding characteristics, as well as fragments and regions thereof.

The antibody of the invention preferably binds anti-IL6 or anti-IL6R with an affinity (K_{d}) of at least 10⁻⁹ M, preferably at least 10⁻¹⁰ M, and/or substantially neutralize at least one activity of at least one IL-6 protein. In a preferred embodiment, the antibody binds IL-6 with an affinity (K_{d}) of at least 1×10⁻¹¹M, preferably 5×10⁻¹¹ neutralizes human IL-6. Preferably, the antibody does not bind other IL-6 superfamily members and blocks trans-signaling of GP130.

### Proteasome Inhibitors

The proteasome is an intracellular structure which is a multicatalytic proteinase which is a highly conserved. Proteasomes are responsible for the ATP-dependent proteolysis of many proteins involved in important regulatory cellular processes. Thus, the proteosome is a regulatory element in cell growth and differentiation. The average human cell contains about 30,000 proteasomes, each of which contains several protein-digesting proteases. These complexes are in a myriad of cellular functions including transcription, cell cycle control, stress response, ribosome biogenesis, and abnormal protein catabolism. Therefore, they play a role in such processes as immune and inflammatory responses (WO 95/25533), viral infection, oncogenesis, neural and muscular degeneration (U.S. Pat. No. 5,340,736), antigen processing (WO 94/17816), DNA repair, and cellular differentiation. Proteasome activity is exquisitely controlled in order to maintain strict governance over the rate and specific types of proteins degraded.

Several steps are involved in protein degradation via the proteasome or “ubiquitin-proteasome” pathway. Initially, a protein is marked for destruction with a chain of small polypeptides known as ubiquitin. Ubiquitinylation guides the protein into the proteosome's enclosed proteolytic chamber. Three enzymatic activities, E1, E2, and E3, are required for ubiquitinylation. The ATP-dependent E1 enzyme activates ubiquitin and links it to the ubiquitin-conjugating enzyme, E2. The E3 enzyme, an ubiquitin ligase, then links the ubiquitin molecule to the protein. This process is repeated until the designated polypeptide trails a long chain of ubiquitin moieties and the proteasome finally degrades the protein into small fragments. The ubiquitin-proteasome pathway is responsible for the degradation of 90% of all abnormal, misfolded proteins and all of the short-lived, regulatory proteins in the cell. These short-lived proteins, whose half-lives are less than three hours, account for 10% to 20% of all cellular proteins. The pathway also breaks down the much of the longer-lived cellular proteins. Thus, the ubiquitin-proteasome pathway is responsible for degrading 80% to 90% of all intracellular proteins.

Early reported proteasome inhibitors included peptidyl aldehydes. Preliminary optimization of these suggested a preference for leucine at the P1 position and a large hydrophobic residue, such as naphthylalanine, at P2 or P3 positions. Because the peptidyl aldehydes also demonstrate potent inhibition of thiol proteases (eg, calpains, cathepsins) and are not configurationally stable due to the acidity of the proton at the alpha-position, replacements for the aldehyde group were investigated.

In addition to antibiotic inhibitors originally isolated from actinomycetes, a variety of peptide aldehydes have been synthesized, such as the inhibitors of chymotrypsin-like proteases described by Siman et al. (WO91/13904). A variety of inhibitors of the proteasome complex have been reported, e.g., Dick, et al., Biochem. 30: 2725 (1991); Goldberg, et al., Nature 357: 375 (1992); Goldberg, Eur. J. Biochem. 203: 9 (1992); Orlowski, Biochem. 29: 10289 (1989); Rivett, et al., Archs. Biochem. Biophys. 218: 1 (1989); Rivett, et al., J. Biol. Chem. 264: 12, 215 (1989); Tanaka, et al., New Biol. 4: 1 (1992). Proteasome inhibitors are also discussed in U.S. Pat. No. 5,693,617, the disclosure of which is incorporated herein by reference.

A preferred proteasome inhibitor is “PS-341” which refers to a boronic acid dipeptide proteasome inhibitor bortezomib,(MLN-341, LDP-341and PS-341; N-(morpholino)carbonyl)-beta-(1-napthyl)-L-alanine-L-leucine boronic acid) sold under the brand name VELCADE®, WO96/013266). PS-341 inhibits the activation of the transcription factor NF-κB. PS-341 also down-regulates the expression of several apoptosis inhibitors, induces caspase-dependent apoptosis of drug resistant multiple myeloma (MM) cell lines and patient cells, inhibits MM cell binding to bone marrow stromal cells (BMSCs) and inhibits production of MM growth and survival factors in the bone marrow milieu.

In contrast to peptide aldehydes, which inhibit activities of both the proteasome and cysteine proteases, bortezomib is a much more potent and selective inhibitor of the proteasome. It has very high selectivity for the proteasome (>500-fold) over other serine proteases, including human leukocyte elastase, cathepsin G, chymotrypsin and thrombin. Bortezomib was recently approved for use to treat relapsed and refractory multiple myeloma. Inhibition of tumor cell proteasome activity by bortezomib in various tumor culture models is associated with induction of apoptosis.

Specific proteasome inhibitors fall into five classes distinguished by the pharmacophore that interacts with the active site threonine in the proteasome: peptide aldehydes such as CEP1612 and MG132, peptide boronates such as bortezomib, peptide vinyl sulfones, peptide epoxyketones and β-lactone inhibitors such as lactocystin. The following compounds, or analogues thereof, are also contemplated to be used as proteasome inhibitors in the present invention: PS-519 (1R-[1S,4R,5S]]-1-(1-hydroxy-2-methylpropyl)-4-propyl-6-oxa-2-azabicyclo[3.2.1.]heptane-3,7-dione); clasto-lactacystin beta-lactone; lactacystin, epoxomicin, CVT634 (-5-methoxy-1-indanone-3-acetyl-leucyl-D-leucyl-1-indanylamide), TMC96 ((3-methylbutanoyl-L-threonine N-(1-(2-(hydroxymethyl)-oxiran-2-ylcarbonyl)-3-methylbut-3enyl)amide), MG-115, CEP1612 and MG132.

In addition to the proteasome protease inhibitors, the ubiquitin-proteasome pathway may be blocked by inhibitors of the facilitating enzymes Ubiquitin-activating enzyme (E1), ubiquitin-conjugating enzyme (E2), and ubiquitin-ligases (E3 enzymes). E1 inhibitors have been identified such as himeic acid A (Tsukamoto, et al. 2005, Bioorgan Med Chem Lett 15(1):191-194. Other methods known in the art, such as RNA silencing, may also be used to reduce or eliminate the activities of specific ubiquitinylation-related enzymes.

### Monitoring of Proteosome Inhibitory Activity

A method for monitoring pharmacodynamic drug action of a proteasome inhibitor in a mammal is taught in U.S. Pat. No. 6,613,541, the inventors having surprisingly discovered that ex vivo assay of proteasome activity, rather than drug concentration, in biological samples provides a useful method for monitoring pharmacodynamic drug action of proteasome inhibitors and that this data provides guidance for selecting a future dose amount and dose frequency of the proteasome inhibitor to be administered in the future.

The method comprises administering the proteasome inhibitor to the mammal; obtaining one or more test biological samples from the mammal at one or more specified times after administering the proteasome inhibitor; measuring proteasome activity in the test biological sample or samples; determining the amount of proteasome activity in the test biological sample or samples; and comparing the amount of proteasome activity in the test biological sample to that in a reference biological sample obtained from a mammal to which no proteasome inhibitor has been administered.

U.S. Pat. No. 6,613,541 further provides a method for determining dose regimen for a proteasome inhibitor, a method for determining baseline proteasome activity in a mammal, including a human, and provides a kit for measuring proteasome activity in a biological sample from a mammal. The methods of U.S. Pat. No. 6,613,541 may be practiced on biological samples selected from a blood, urine, and tissue biopsy sample.

### Measurement of IL6

IL6 can be detected in bioassays employing IL6 responsive cell lines (see: 7TD1; B9; CESS, KPMM2, KT-3; M1, MH60-BSF-2, MO7E; Mono Mac 6; NFS-60; PIL-6; SKW6-C14; T1165; XG-1). IL6 can be assayed also by its activity as a hybridoma growth factor (see: HGF). Sensitive immunoassays and colorimetric tests are also available. An alternative detection method is RT-PCR quantitation of cytokines . An ELISA assay exists for detecting the receptor-associated gp130 protein (such reagents are available from e.g. R&D Systems).

For detection of IL6 bound to CNTO328, the anti-ID (anti-variable region antibodies disclosed in applicants copending applications U.S. Ser. No. 10/280716 may be used to detect in any standard immunoassay format such as an ELISA-type assay.

### Diseases Amenable to Treatment by the Method of the Invention

The deregulated expression of IL6 is probably one of the major factors involved in the pathogenesis of a number of diseases. The excessive overproduction of IL6 (and other B-cell differentiation factors) has been observed in various pathological conditions such as rheumatoid arthritis, multiple myeloma, Lennert syndrome (histiocytic lymphoma), Castleman's disease (lymphadenopathy with massive infiltration of plasma cells, hyper gamma-globulinemia, anemia, and enhanced concentrations of acute phase proteins), cardiac myxomas and liver cirrhosis. Constitutive synthesis of IL6 by glioblastomas and the secretion of IL6 into the cerebrospinal fluid has been observed.

With respect to immune mediated inflammatory diseases (IMIDs), IL6 is implicated in the pathogenesis of chronic polyarthritis (together with IL1 and IL8) since excessive concentrations of IL6 are found in the synovial fluid. In inflammatory intestinal diseases elevated plasma levels of IL6 may be an indicator of disease status. In patients with mesangial proliferative glomerulonephritis elevated urine levels of IL6 are also an indicator of disease status. IL6 may play a role in the immune mediated pathogenesis of diabetes mellitus of both type I and type II.

Accordingly, the present invention also provides a method for modulating or treating at least one IL-6 related disease, in a cell, tissue, organ, animal, or patient, as known in the art or as described herein, using at least one IL-6 antibody of the present invention, e.g., administering or contacting the cell, tissue, organ, animal, or patient with a therapeutic effective amount of IL-6 antibody in conjunction with administration of a proteasome inhibitor. The present invention also provides a method for modulating or treating at least one IL-6 related disease, in a cell, tissue, organ, animal, or patient including, but not limited to, at least one of obesity, an immune related disease, a cardiovascular disease, an infectious disease, a malignant disease or a neurologic disease.

The present invention also provides a method for modulating or treating at least one IL-6 related immune related disease, in a cell, tissue, organ, animal, or patient including, but not limited to, at least one of rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, osteolysis, aseptic loosening of orthopedic implants, inflammatory bowel disease, ulcerative colitis, systemic lupus erythematosus, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, burns, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, diabetes, nephrosis, atopic diseases, hypersensitity reactions, allergic rhinitis, hay fever, perennial rhinitis, conjunctivitis, endometriosis, asthma, urticaria, systemic anaphalaxis, dermatitis, pernicious anemia, hemolytic disease, thrombocytopenia, graft rejection of any organ or tissue, kidney transplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, asthma, myasthenia gravis, antibody-meditated cytotoxicity, type III hypersensitivity reactions, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, diabetes mellitus, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, diabetic retinopathy, hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, okt3 therapy, anti-cd3 therapy, cytokine therapy, chemotherapy, radiation therapy (e.g., including but not limited to, asthenia, anemia, cachexia, and the like), chronic salicylate intoxication, and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2000), each entirely incorporated by reference.

The present invention also provides a method for modulating or treating at least one cardiovascular disease in a cell, tissue, organ, animal, or patient, including, but not limited to, at least one of cardiac stun syndrome, myocardial infarction, congestive heart failure, stroke, ischemic stroke, hemorrhage, arteriosclerosis, atherosclerosis, restenosis, diabetic ateriosclerotic disease, hypertension, arterial hypertension, renovascular hypertension, syncope, shock, syphilis of the cardiovascular system, heart failure, cor pulmonale, primary pulmonary hypertension, cardiac arrhythmias, atrial ectopic beats, atrial flutter, atrial fibrillation (sustained or paroxysmal), post perfusion syndrome, cardiopulmonary bypass inflammation response, chaotic or multifocal atrial tachycardia, regular narrow QRS tachycardia, specific arrythmias, ventricular fibrillation, His bundle arrythmias, atrioventricular block, bundle branch block, myocardial ischemic disorders, coronary artery disease, angina pectoris, myocardial infarction, cardiomyopathy, dilated congestive cardiomyopathy, restrictive cardiomyopathy, valvular heart diseases, endocarditis, pericardial disease, cardiac tumors, aordic and peripheral aneuryisms, aortic dissection, inflammation of the aorta, occlusion of the abdominal aorta and its branches, peripheral vascular disorders, occlusive arterial disorders, peripheral atherlosclerotic disease, thromboangitis obliterans, functional peripheral arterial disorders, Raynaud's phenomenon and disease, acrocyanosis, erythromelalgia, venous diseases, venous thrombosis, varicose veins, arteriovenous fistula, lymphederma, lipedema, unstable angina, reperfusion injury, post pump syndrome, ischemia-reperfusion injury, and the like. Such a method can optionally comprise administering an effective amount of a composition or pharmaceutical composition comprising at least one anti-IL-6 antibody to a cell, tissue, organ, animal or patient in need of such modulation, treatment or therapy.

The present invention also provides a method for modulating or treating at least one IL-6 related infectious disease in a cell, tissue, organ, animal or patient, including, but not limited to, at least one of: acute or chronic bacterial infection, acute and chronic parasitic or infectious processes, including bacterial, viral and fungal infections, HIV infection/HIV neuropathy, meningitis, hepatitis (e.g., A, B or C, or the like), septic arthritis, peritonitis, pneumonia, epiglottitis, e. coli 0157:h7, hemolytic uremic syndrome/thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis/epidydimitis, legionella, lyme disease, influenza a, epstein-barr virus, viral-associated hemaphagocytic syndrome, viral encephalitis/aseptic meningitis, and the like.

The present invention also provides a method for modulating or treating at least one IL-6 related malignant disease in a cell, tissue, organ, animal or patient, including, but not limited to, at least one of: leukemia, acute leukemia, acute lymphoblastic leukemia (ALL), acute lymphocytic leukemia, B-cell, T-cell or FAB ALL, acute myeloid leukemia (AML), acute myelogenous leukemia, chromic myelocytic leukemia (CML), chronic lymphocytic leukemia (CLL), hairy cell leukemia, myelodyplastic syndrome (MDS), a lymphoma, Hodgkin's disease, a malignant lymphoma, non-hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometiral cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

The present invention also provides a method for modulating or treating at least one IL-6 related neurologic disease in a cell, tissue, organ, animal or patient, including, but not limited to, at least one of: neurodegenerative diseases, multiple sclerosis, migraine headache, AIDS dementia complex, demyelinating diseases, such as multiple sclerosis and acute transverse myelitis; extrapyramidal and cerebellar disorders, such as lesions of the corticospinal system; disorders of the basal ganglia; hyperkinetic movement disorders, such as Huntington's Chorea and senile chorea; drug-induced movement disorders, such as those induced by drugs which block CNS dopamine receptors; hypokinetic movement disorders, such as Parkinson's disease; Progressive supranucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi-system disorder); demyelinating core disorders, such as multiple sclerosis, acute transverse myelitis; and disorders of the motor unit' such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Hallerrorden-Spatz disease; Dementia pugilistica; neurotraumatic injury (e.g., spinal cord injury, brain injury, concussion, repetitive concussion); pain; inflammatory pain; autism; depression; stroke; cognitive disorders; epilepsy; and the like. Such a method can optionally comprise administering an effective amount of a composition or pharmaceutical composition comprising at least one TNF antibody or specified portion or variant to a cell, tissue, organ, animal or patient in need of such modulation, treatment or therapy. See, e.g., the Merck Manual, 16^{th} Edition, Merck & Company, Rahway, N.J. (1992).

### Methods of Administration

The method of the present invention comprises administering an effective amount of a composition or pharmaceutical composition comprising at least one anti-IL-6 antibody to a cell, tissue, organ, animal or patient in need of such modulation, treatment or therapy in conjunction with treatment comprising administration of a proteasome inhibitor. The method of the invention comprises treating such diseases or disorders, wherein the administering of said at least one IL-6 antagonist is indicated. The method of the invention further comprises the co-administration with the IL6 antagonist, before, concurrently, and/or after, at least one proteasome inhibitor. In a specific embodiment, the IL6 antagonist is an antibody which prevents or inhibits the biological functions of IL6, such as a neutralizing IL6 antibody or an anti-IL6R antibody, and the proteasome inhibitor is selected from the group consisting of PS-314 (bortezomib), PS-519; clasto-lactacystin beta-lactone; lactacystin, epoxomicin, CVT634, TMC96, MG-115, CEP1612 and MG132.

Typically, treatment of pathologic conditions is effected by administering an effective amount or dosage of an anti-IL-6 antibody composition that total, on average, a range from at least about 0.01 to 500 milligrams of at least one anti-IL-6 antibody per kilogram of patient per dose, and, preferably, from at least about 0.1 to 100 milligrams antibody/kilogram of patient per single or multiple administration, depending upon the specific activity of the active agent contained in the composition. Alternatively, the effective serum concentration can comprise 0.1-5000 microgm/ml serum concentration per single or multiple administrations. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

For parenteral administration, the antibody or the proteasome inhibitor can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Administration

Many known and developed modes can be used according to the present invention for administering pharmaceutically effective amounts of the IL6 antagonist and proteasome inhibitor according to the present invention. While parenteral administration is typical, other modes of administration can be used according to the present invention with suitable results. Composition of the present invention can be delivered in a carrier, as a solution, emulsion, colloid, or suspension, or as a dry powder, using any of a variety of devices and methods suitable for administration by inhalation or other modes described here within or known in the art.

Alternative routes of administration include subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means.

In accordance with the invention, the combination IL6 antagonist and proteasome inhibitor and further optionally include one or more agents commonly used in treating IL-6 related conditions as discussed above. Such agents include, for example a corticosteriod (dexamethasone), a topoisomerase inhibitor (etoposide, irinotecan), a cytoxin (doxorubicin), an alkylating agent (carboplatin), a nitrogen mustard (melphalen, chlorabucil), a nitrosourea (carmustine, estramustine) an antimetabolite (methotrexate, cytarabine, fluorouracil), a mitotic inhibitor (vincristine, taxol), a radiopharmaceutical (Iodine131-tositumomab), a radiosensitizer (misonidazole, tirapazamine), a cytokine (interferon alpha-2, IL2) or a cytokine antagonist (inflixamab). Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

### EXAMPLE 1

### Activity of ANTI-IL6 Antibody in Combination with a Proteasome Inhibitor

Based on the hypothesis that inhibition of IL-6 with the chimeric, monoclonal IL-6-neutralizing antibody, CNTO 328, would potentiate the anti-myeloma activity of the proteasome inhibitor, bortezomib, by attenuating bortezomib-mediated up-regulation of HSP-70, the following studies were designed and executed.

A. Inhibition of IL-6 Signaling with the Monoclonal Antibody, CNTO 328, Decreases Cell Viability of the IL-6-Dependent Multiple Myeloma Cell Lines, ANBL-6 and KAS-6.

ANBL-6 and KAS-6 cells (Dr. Diane Jelinek, Mayo Clinic, Rochester, Minn.) were incubated with increasing concentrations of CNTO 328 or the isotype control antibody, F105, for 24 and 48 hours. For the last 4 hours, the cells were incubated in the presence of WST-1 (Roche Applied Science, Indianapolis, Ind.). Reduction of WST-1 into a water soluble formazan salt by viable cells was measured at an absorbance of 450 nM using an ELISA plate reader. Viability was measured as percent viability relative to untreated cells. All cells were treated in RPMI 1640 media containing 10% FBS and 1 ng/mL of IL-6.

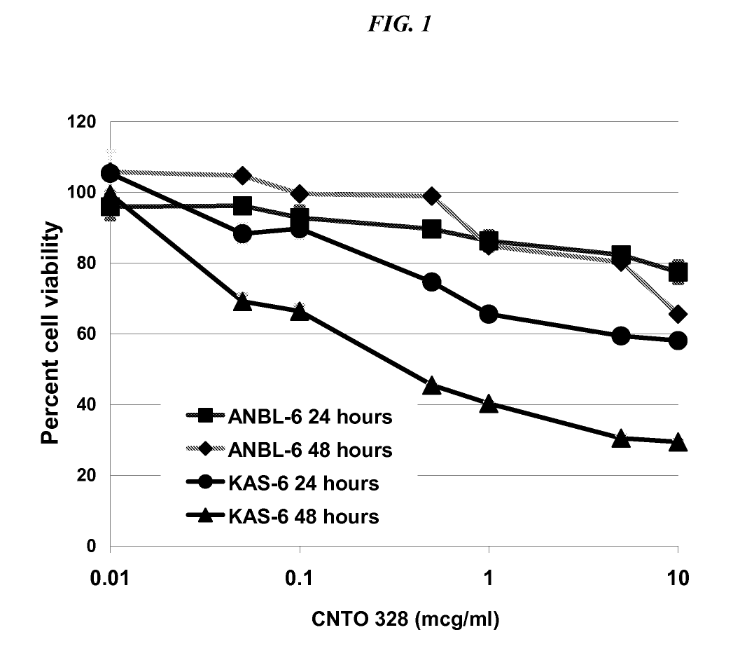

The results are shown graphically in FIG. 1 (values are the mean of quintuplicate cultures; bars, SEM). Treatment of ANBL-6 and KAS-6 cells with CNTO 328 leads to a dose- and time-dependent reduction in cell viability. KAS-6 cells were significantly more sensitive to the effects of IL-6 inhibition than ANBL-6 cells.

### B. CNTO 328 Potentiates the Anti-Myeloma Activity of Bortezomib in IL-6-Dependent Myeloma Cells.

The ANBL-6, KAS-6, or IL-6-independent RPMI 8226 myeloma cell lines were pre-incubated with 0.1 mcg/ml (KAS-6) or 10 mcg/ml (ANBL-6 and RPMI 8226) of the control antibody, F105, or CNTO 328 for 24 hours, followed by co-incubation with either DMSO control or increasing concentrations of bortezomib in the continued presence of F105 or CNTO 328 for another 24 hours. For sequencing experiments, ANBL-6 cells were treated with: 1) F105 or CNTO 328 for 24 hours followed by F105 or CNTO 328 and bortezomib 5 nM for another 24 hours (antibody→bortezomib); 2) F105 or CNTO 328 and bortezomib 5 nM concurrently for 24 hours (antibody+bortezomib), or bortezomib at 5 nM for 12 hours followed by F105 or CNTO 328 for 24 hours (bortezomib→antibody). Cell viability was assayed as described above and measured as percent viability relative to untreated cells. All cells were treated in RPMI 1640 media containing 10% FBS and 1 ng/mL of IL-6. Columns, mean of quintuplicate cultures; bars, SEM.

Pre-incubation of ANBL-6 and KAS-6 cells with CNTO 328 potentiated the cytotoxicity of bortezomib, as demonstrated by a significant reduction in cell viability relative to cells pre-treated with the control antibody, F105 (FIGS. 12 A and B). CNTO 328 did not enhance the activity of bortezomib in the IL-6-independent myeloma cell line, RPMI 8226 (panel C). CNTO 328 enhanced the cytotoxicity of bortezomib most when ANBL-6 cells were pre-treated with CNTO 328 followed by bortezomib or treated concurrently with CNTO 328 and bortezomib. In contrast, CNTO 328 had little additional effect when cells were pre-treated with bortezomib (panel D).

C. Inhibition of IL-6 Signaling with CNTO 328 Enhances Bortezomib-Mediated Apoptosis of the IL-6-Dependent Multiple Myeloma Cell Lines, ANBL-6 and KAS-6.

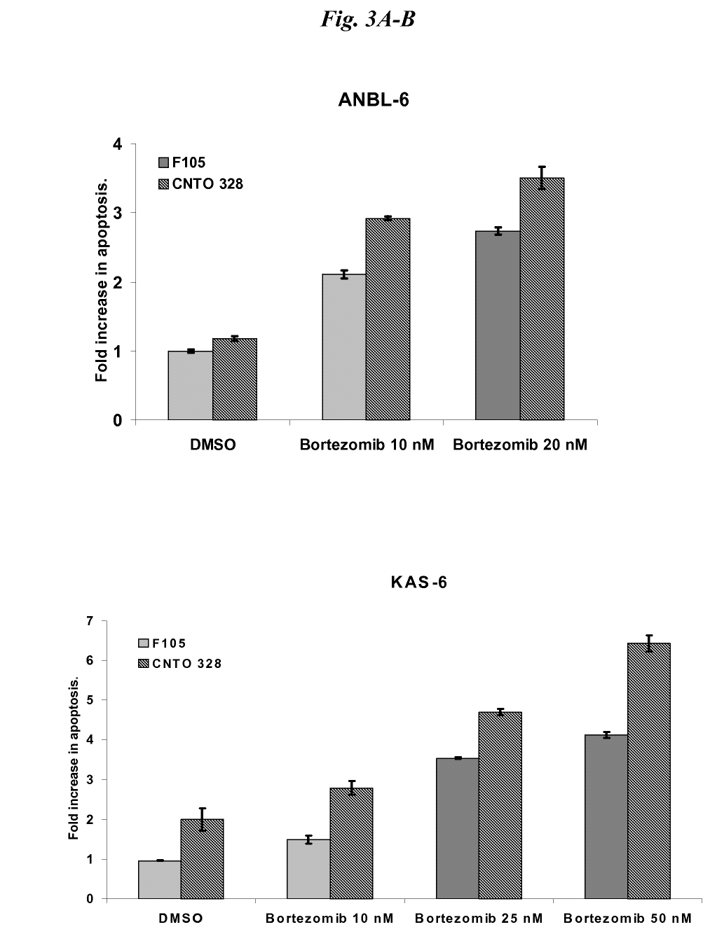

ANBL-6 and KAS-6 cells were incubated with 10 mcg/ml (ANBL-6) or 1 mcg/ml (KAS-6) of CNTO 328 or the control antibody, F105, for 8 (ANBL-6) to 12 (KAS-6) hours. Apoptosis was determined using an ELISA-based assay that measures the presence of mono- and oligo-nucleosomes (Roche Applied Science, Indianapolis, Ind.) and expressed as a fold increase in apoptosis over DMSO and F105-treated controls. Cells were treated in RPMI 1640 media containing 10% FBS and 1 ng/mL of IL-6 (FIGS. 3 A and B, column height represents the mean of triplicate cultures; bars, SEM).

Treatment of ANBL-6 and KAS-6 cells with CNTO 328 and bortezomib led to enhanced induction of apoptosis compared with cells treated with either drug alone. CNTO 328 was not able to potentiate apoptosis in the IL-6-independent myeloma cell line RPMI 8226 (data not shown).

### D. CNTO 328 Down-Regulates Interleukin-6 Signaling and Attenuates Bortezomib-Mediated Induction of Anti-Apoptotic MKP-1 and HSP-70 in ANBL-6 Cells.

ANBL-6 cells were incubated with 10 mcg/ml of CNTO 328 or the control antibody, F105, with or without increasing concentrations of bortezomib for 8 hours. Cell lysates were prepared and subjected to immunoblot analysis. Blots were stripped and probed for HSC-70 to ensure equal protein loading per lane. Densitometry was performed on HSP-70 and MKP-1 immunoblots.

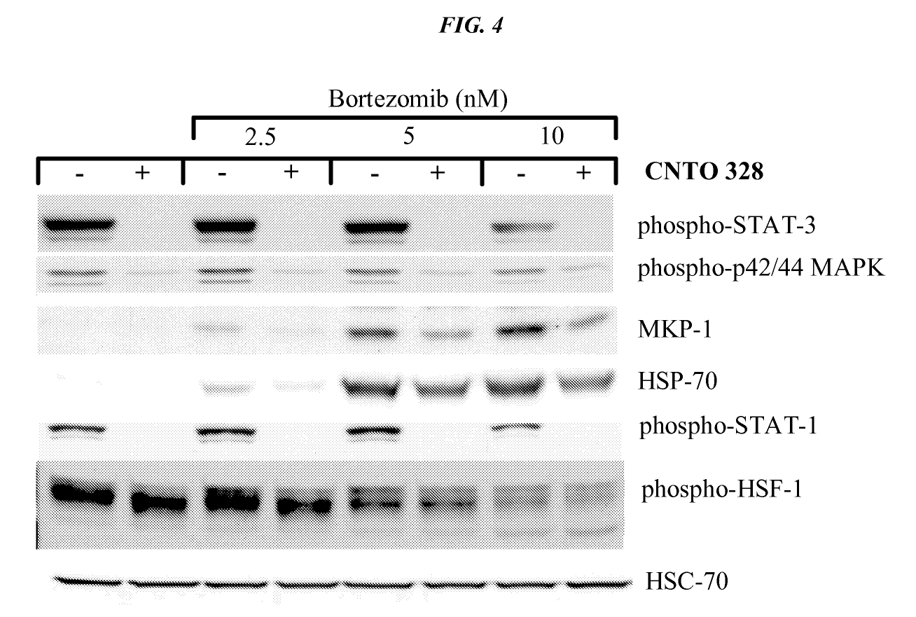

Treatment of ANBL-6 cells with CNTO 328 led to a dramatic decrease in downstream mediators of IL-6 signaling as demonstrated by a reduction in levels of phospho-p42/44 MAPK and phospho-STAT-3 (FIG. 4). Notably, increasing doses of bortezomib also decreased levels of phospho-STAT-3 and phospho-p42/44 MAPK. These data indicate that ortezomib, interferes with IL-6 signaling (also reported Hideshima T et al, Oncogene 2003, 22: 8386-93) Furthermore, CNTO 328 interfered with bortezomib-mediated induction of HSP-70 and MKP-1 by 45 and 90%, respectively, which correlated with decreased levels of transcriptionally active phospho-STAT-1 and hyperphosphorylated HSF-1.

### E. KNK437 Enhances Bortezomib-Mediated Apoptosis of ANBL-6 and HSF-1+/+MEF Cells.

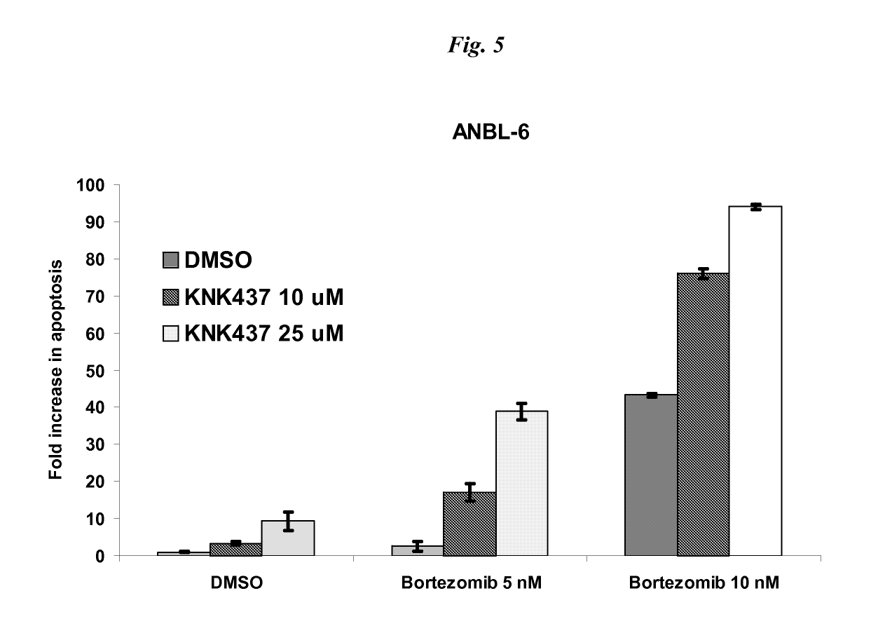

ANBL-6 or MEF cells (control and HSF-1−/−) were incubated with either DMSO control or increasing concentrations of bortezomib and KNK437 for 12 (ANBL-6) to 24 hours (MEFs). Apoptosis was determined as described above and expressed as a fold increase in apoptosis over DMSO-treated controls. ANBL-6 cells were treated in RPMI 1640 media containing 10% FBS and 1 ng/mL of IL-6 (FIG. 5, columns, mean of triplicate cultures; bars, SEM).

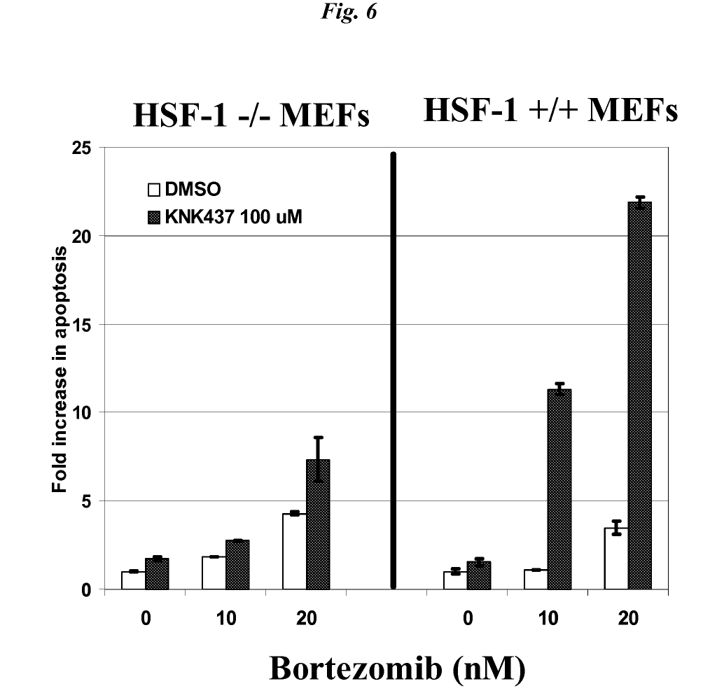

Treatment of ANBL-6 cells with KNK437 led to a significant increase in bortezomib-mediated apoptosis compared with control treated cells. The enhancement of bortezomib-mediated apoptosis was blunted in HSF-1−/−MEFs relative to control MEFs (FIG. 6), suggesting that the increased activity of the combination is due to down-regulation of the heat shock protein response.

Summary of Results of CNTO328 Combination with Bortezomib

The IL-6 neutralizing antibody CNTO 328 decreased viability of the multiple myeloma cell lines ANBL-6 and KAS-6 in a dose- and time-dependent manner.

Treatment of ANBL-6 and KAS-6 cells with CNTO 328 potentiated the anti-myeloma activity of bortezomib as demonstrated by a reduction in cell viability and enhancement of apoptosis with the combination compared with a control antibody and bortezomib. The anti-myeloma effect of CNTO 328 was diminished when cells were treated sequentially with bortezomib followed by CNTO 328 rather than the reverse order, perhaps due to the ability of bortezomib to down-regulate important downstream mediators of IL-6 signaling, or by earlier up-regulation of members of the heat shock protein response.

Treatment of ANBL-6 and KAS-6 cells with CNTO 328 led to down-regulation of IL-6 signaling as shown by a marked decrease in phospho-STAT-3 and phospho-p42/44 MAPK levels. Bortezomib also down-regulated phospho-STAT-3 and phospho-p42/44 MAPK levels in a concentration-dependent manner.

The increased activity of the combination of CNTO 328 and bortezomib was associated with decreased bortezomib-mediated accumulation of anti-apoptotic HSP-70 and MKP-1. Decreased HSP-70 induction correlated with decreased levels of phospho-STAT-1 and hyperphosphorylated HSF-1.

Treatment of ANBL-6 and KAS-6 cells with KNK437 enhanced the apoptotic activity of bortezomib, which in part was due to its ability to interfere with induction of the heat shock protein response, as demonstrated by the fact that the increased apoptotic effect was markedly blunted in HSF-1-negative mouse embryonic fibroblasts.

Taken together, the above data provide a rationale for translating the bortezomib/CNTO 328 combination into clinical trials and devising other novel strategies aimed at down-regulating resistance to bortezomib (e.g. inhibitors of HSP-70, MKP-1).

What is claimed is:

1. A method of treating a cancerous disorder or condition in a mammal in need of such treatment which comprises co-administering a proteasome inhibitor in combination with an IL-6 antagonist.

2. The method according to claim 1, in which the IL-6 antagonist is an antibody or a fragment thereof.

3. The method according to claim 2 in which the antibody is a monoclonal antibody.

4. The method according to claim 2, in which the antibody or fragment binds to IL6.

5. The method according to claim 2 in which the antibody or fragment binds to the IL-6 receptor.

6. The method according to claims 3 or 4, in which the antibody fragment is an Fab, Fab′, or F(ab′)2 fragment or derivative thereof.

7. The method according to claim 3, in which the monoclonal antibody competes with monoclonal antibody cCLB8 for binding to human IL6.

8. The method according to claim 3, in which the monoclonal antibody is administered intravenously

9. The method according to claim 3, in which the monoclonal antibody is administered in the amount of from 0.01 mg/kg to 12.0 mg/kg body weight.

10. The method according to claim 3, in which the monoclonal antibody is administered in a bolus dose followed by an infusion of said antibody.

11. The method according to claim 1, in which the mammal is a human patient.

12. The method according to claim 1 in which the proteasome inhibitor is selected from the group consisting of the boronic acid dipeptide proteasome inhibitor bortezomib, PS-519 (1R-[1S,4R,5S]]-1-(1-hydroxy-2-methylpropyl)-4-propyl-6-oxa-2-azabicyclo[3.2.1.]heptane-3,7-dione); clasto-lactacystin beta-lactone; lactacystin, epoxomicin, CVT634 (-5-methoxy-1-indanone-3-acetyl-leucyl-D-leucyl-1-indanylamide), TMC96 ((3-methylbutanoyl-L-threonine N-(1-(2-(hydroxymethyl)-oxiran-2-ylcarbonyl)-3-methylbut-3enyl)amide), MG-115, CEP1612 and MG132.

13. The method according to claim 1 in which the proteasome inhibitor is the boronic acid dipeptide proteasome inhibitor bortezomib.

14. The method according to claim 1, in which the cancerous disorder or condition is at least one selected from leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), hairy cell leukemia, myelodyplastic syndrome (MDS), a lymphoma, Hodgkin's disease, a malignant lymphoma, non-hodgkin's lymphoma, Burkitt's lymphoma, multiple myeloma, Kaposi's sarcoma, colorectal carcinoma, pancreatic carcinoma, renal cell carcinoma, prostatic cell carcinoma, nasopharyngeal carcinoma, malignant histiocytosis, paraneoplastic syndrome/hypercalcemia of malignancy, solid tumors, adenocarcinomas, sarcomas, and malignant melanoma.

15. The method according to claim 1 in which the anti-IL6 antagonist is administered sequentially, serially, or concurrently with the proteosome inhibitor.

16. A method for inhibiting tumor growth in a mammal in need thereof comprising administering to the mammal in conjunction with a proteasome inhibitor, a monoclonal antibody or fragment thereof which prevents IL6 activation of signaling through membrane bound receptors in an amount effective to inhibit the growth of said tumor.

17. A method for preventing metastases in a mammal comprising administering to the mammal in conjunction with a proteasome inhibitor, a monoclonal antibody or fragment thereof which prevents IL6 activation of signaling through membrane bound receptors in an amount effective to prevent metastases in said mammal.

18. A method of any of claims 3, 16 or 17 wherein the antibody is cCLB8 or a fragment thereof.

19. A method of treating an IL-6 related disorder or condition, in a mammal in need of such treatment, which comprises co-administering a proteasome inhibitor in combination with an IL-6 antagonist.
